(19) United States
(12) Patent Application Publication
(10) Pub. No.: US 2026/0110009 A1
(43) Pub. Date: Apr. 23, 2026

(54) PROCESS FOR CONTINUOUS MANUFACTURE OF BIOMOLECULES

(71) Applicants: Technische Universität Clausthal, CLAUSTHAL-ZELLERFELD (DE); Dillico, Meylan (FR)

(72) Inventors: Jochen Strube, Göttingen (DE); Axel Schmidt, Clausthal-Zellerfeld (DE); Heribert Helgers, Goslar (DE); Alina Hengelbrock, Clausthal-Zellerfeld (DE); Christophe Bonneville, Saint-Nazaire-Les-Eymes (FR); Florian Vetter, Clausthal-Zellerfeld (DE); Alex Juckers, Clausthal-Zellerfeld (DE)

(21) Appl. No.: 19/115,401

(22) PCT Filed: Sep. 27, 2023
(86) PCT No.: PCT/EP2023/076748

§ 371 Date: Mar. 26, 2025

(30) Foreign Application Priority Data
Sep. 30, 2022 (EP) 22315224.0

(51) Int. Cl. C12P 19/34; C12M 1/00; C12M 1/02; C12P 21/00

(52) U.S. Cl. CPC C12P 19/34; C12M 27/00; C12M 47/12; C12P 21/00

(57) ABSTRACT

A system for continuous manufacture of biomolecules includes feed tanks suitable for storing products and a reaction chamber designed to be fed with products from the feed tanks that form a reaction phase in the reaction chamber. The reaction chamber is designed to manufacture the biomolecules from the reaction phase. The system includes a scale tank in fluid communication with the reaction chamber, which is suitable for storing an immiscible phase, and a monitoring and control unit set to control the injection flow rate of the immiscible phase into the reaction chamber to keep constant a certain filling level of the reaction chamber according to the amount of reaction phase to be injected into the reaction chamber to manufacture a certain amount of biomolecules.

### CROSS-REFERENCE TO RELATED APPLICATIONS

This application is a national phase entry under 35 U.S.C. § 371 of International Patent Application PCT/EP2023/076748, filed Sep. 27, 2023, designating the United States of America and published as International Patent Publication WO 2024/068754 A1 on Apr. 4, 2024, which claims the benefit under Article 8 of the Patent Cooperation Treaty of European Patent Application Serial No. 22315224.0, filed Sep. 30, 2022.

### TECHNICAL FIELD

The present disclosure generally relates to the field of in vitro production of biomolecules, and, in particular, the production of mRNA into lipid nanoparticles for use in therapeutic applications, such as vaccines.

### BACKGROUND

Scientific and technological advances of the recent years have made biomolecules promising candidates for a variety of uses, including diagnostic applications, and therapeutic products, like vaccines.

Propelled by major science-technology-medicine advancements, the rise of new therapies that target the genetic machinery, such as gene replacement, correction or modulation, and protein expression, is enabling new opportunities for innovative treatment, but also emergency responses in epidemic crisis situations. In that context, various approaches have been developed for mRNA production at scale. Most current processes utilize in vitro enzymatic reaction to synthesize mRNA from self-replicating DNA templates, then encapsulate the total RNA into a lipid nanoparticle as an immiscible delivery vehicle.

These processes are costly, require highly skilled personnel, produce hazardous waste streams that must be mediated, while the production rate is severely dependent on the performance of the enzymatic reactions, the ability to remove process and product related impurities and the control and performance of the encapsulation into lipid nanoparticles.

In addition, the current practice is to produce mRNA in batch mode, which gives little flexibility in terms of changing the scale of the production: for each scale, the process has to be significantly adapted and changes have to be approved by regulatory authorities.

The recent pandemic has shown that there is a need to accelerate the development of new vaccines and their availability to the population.

In this context, WO2021212034 has described a novel process to produce mRNA in an in vitro system using a continuous flow production.

The system described in WO2021212034 has a reaction chamber in which in vitro transcription is performed to continuously produce mRNA.

The reaction chamber operates continuously such that it may be infused with new input material while producing mRNA.

This system is particularly adapted for producing a large amount of mRNA, such as millions of doses in the context of a pandemic, but will not be adapted to produce a small quantity of mRNA, for example, thousands of doses for early clinical trials, without wasting a large quantity of enzymes and buffers.

Indeed, in the context of changing scale-up in mRNA production, the production of a small quantity would generally be done in a smaller batch reactor with a volume adapted to the desired amount of output.

Thus, the system described in WO2021212034 is not adapted to rapidly elaborate and manufacture different quantities of mRNA with a reduced cost.

### BRIEF SUMMARY

The objective of the present disclosure is therefore to solve the above-mentioned problems.

To this end, the present disclosure relates to a system for continuous manufacture of biomolecules comprising feed tanks suitable for storing products and a reaction chamber designed to be fed with products from the feed tanks, which form in the reaction chamber a reaction phase, the reaction chamber being designed to manufacture the biomolecules from the reaction phase, wherein the system comprises a scale tank in fluid communication with the reaction chamber and suitable for storing an immiscible phase, which is not miscible with the reaction phase, the system comprising a monitoring and control unit set to control the injection flow rate of the immiscible phase into the reaction chamber in order to maintain a certain filling level of the reaction chamber according to the amount of reaction phase to be injected into the reaction chamber to manufacture a certain amount of biomolecules, so that the lower the amount of reaction phase to be injected into the reaction chamber and the higher the amount of immiscible phase to be injected.

The idea behind the present disclosure is to use a single system adapted to manufacture from small quantity of biomolecules to a large quantity of biomolecules, or the reverse.

The idea involves, more particularly, dimensioning the system for the manufacture of biomolecules on a large scale while allowing the manufacture of biomolecules on a small scale by preserving the same conditions of reaction and thus the same quality of results (concentration, purity, time of manufacture, etc.), i.e., that the reaction phase must always have the same conditions of reaction.

To this end, an immiscible phase is injected to complete the volume not used by the reaction phase in the reaction chamber in case of small quantity to be manufactured even for scale lower than what the reaction chamber has been typically designed for.

The two phases being immiscible, the reaction phase running in the reaction chamber is always contained in a volume adapted for a stable and effective reaction.

The system of the present disclosure is thus advantageously adapted to produce different quantities of biomolecules, for example, at different stages of vaccine clinical development.

The idea is also to use a single system to produce a chosen quantity of biomolecules in a certain constant residence time. This means that the system according to the present disclosure is designed to produce small or large quantities of biomolecules in the same residence time.

The system according to the present disclosure may also have the following features:
the system comprises a valve at the outlet of the reaction chamber and the monitoring and control unit is set to receive data representative of the presence of the reaction phase at the outlet of the reaction chamber and to control in turn the valve for separating the immiscible phase from the reaction phase;
the data representative of the presence of the reaction phase at the outlet of the reaction chamber is calculated based on a duration of a residence time of the reaction phase in the reaction chamber;
the system comprises a mixing chamber in fluid communication between the feed tanks and the reaction chamber, the mixing chamber being designed to be fed with products from the feed tanks and to perform a mixing of the products to form the reaction phase and to feed in turn the reaction phase to the reaction chamber, the monitoring and control unit being set to control the injection flow rate of the products from each feed tank in real time in order to maintain a constant ratio between the products in the mixing chamber;
the system comprises a first chromatography device comprising a first column in fluid communication between the reaction chamber and a collection tank and a second column in fluid communication between the first column and the collection tank, the first chromatography device comprising a first temporary storage tank in fluid communication between the first column and the second column, the first chromatography device further comprising a first outlet valve at the outlet of the first column designed to switch the fluid communication from the first column to the first temporary storage tank or from the first column to the collection tank, and the first chromatography device comprising an first inlet valve at the inlet of the second column designed to open or close the fluid communication between the first temporary storage tank and the second column;
the first chromatography device comprises a second temporary storage tank in fluid communication between the second column and the first column, the first chromatography device comprising a second outlet valve at the outlet of the second column designed to switch the fluid communication from the second column to the second temporary storage tank or from the second column to the collection tank, the first chromatography device further comprising a second inlet valve at the inlet of the first column designed to open or close the fluid communication from the second temporary storage tank to the first column;
the first chromatography device comprises means for measuring the concentration of biomolecules at the outlet of the first or second columns and the monitoring and control unit is set to control the outlet valve at the outlet of the first or second columns in order to feed, respectively, the first or second temporary storage tanks when the concentration of biomolecules in pre and post fractions of an eluted product fraction from the first or second column is above a certain predetermined concentration threshold or to feed a waste tank when the concentration of biomolecules is below the certain predetermined concentration threshold;
the system comprises a scale buffer tank suitable for storing a completing buffer, the scale buffer tank being fluidly connected to the first column, and the monitoring and control unit is set to control the injection flow rate of the completing buffer from the scale buffer tank into the first column in order to maintain a certain filling level of the column according to the amount of reaction phase to be injected into the column, so that the lower the amount of reaction phase to be injected into the first column and the higher the amount of buffer to be injected into the first column;
the system comprises an inline diafiltration system in fluid communication between the purification device and a lipid nanoparticles formulation system, the diafiltration system comprising several stages designed to pass the reaction phase with an exchange buffer, the monitoring and control unit is set to receive data representative of a certain concentration of biomolecules to be used by the lipid nanoparticles formulation system to form the lipid nanoparticles, the monitoring and control unit being set to control the injection of the exchange buffer in the last stage in order to dilute the biomolecules at the certain concentration of biomolecules;
the system comprises a purification system arranged at the outlet of the reaction chamber designed to separate the manufactured biomolecules from the rest of the reaction phase;
the system comprises means for measuring a certain concentration of each product in the rest of the reaction phase and the monitoring and control unit is set to control the injection of the rest of the reaction phase in the mixing chamber.

The present disclosure also extends to a process for continuous manufacture of biomolecules with the system of the present disclosure, wherein the manufactured biomolecules are RNA, such as mRNA, or proteins or DNA, and wherein the manufactured biomolecules could be a therapeutic agent, such as a vaccine.

More particularly, the process of the present disclosure for continuous manufacturing of biomolecules comprises the following steps:
feeding a reaction chamber with products from feed tanks, the products forming in the reaction chamber a reaction phase, manufacturing the biomolecules from the reaction phase in the reaction chamber, storing an immiscible phase that is not miscible with the reaction phase in a scale tank in fluid communication to the reaction chamber, and controlling with a monitoring and control unit the injection flow rate of the immiscible phase into the reaction chamber in order to maintain a certain filling level of the reaction chamber according to the amount of reaction phase to be injected into the reaction chamber to manufacture a certain amount of biomolecules, so that the lower the amount of reaction phase to be injected into the reaction chamber and the higher the amount of immiscible phase to be injected.

The process of the present disclosure may also comprise the following steps:
receiving in the monitoring and control unit data representative of the presence of the reaction phase at the outlet of the reaction chamber, and controlling in turn with the monitoring and control unit a valve at the outlet of the reaction chamber for separating the immiscible phase from the reaction phase;
the data representative of the presence of the reaction phase at the outlet of the reaction chamber is calculated based on a duration of a residence time of the reaction phase in the reaction chamber;
feeding a mixing chamber in fluid communication between the feed tanks and the reaction chamber with products from the feed tanks, performing a mixing of the products to form the reaction phase and feeding in turn the reaction phase to the reaction chamber, and controlling with the monitoring and control unit the injection flow rate of the products from each feed tank in real time in order to maintain a constant ratio between the products in the mixing chamber;
performing chromatography steps involving separating a product fraction eluted from a first column to a pre and post fractions of the product fraction eluted from the first column;
directing the pre and post fractions from the first column to a first temporary storage tank, and directing the pre and post fraction from the first temporary storage tank to a second column once both pre and post fractions are stored in the temporary storage tank;
separating a product fraction eluted from the second column to a pre and post fractions of the product fraction eluted from the second column, and directing the pre and post fractions from the second column to a second temporary storage tank, and directing the pre and post fraction from the second temporary storage tank to the first column once both pre and post fractions are stored in the second temporary storage tank;
measuring the concentration of biomolecules at the outlet of the first or second columns, and controlling with the monitoring and control unit the outlet valve at the outlet of the first or second columns in order to feed, respectively, the first or second temporary storage tanks when the concentration of biomolecules in pre and post fractions of an eluted product fraction from the first or second column is above a certain predetermined concentration threshold or to feed a waste tank when the concentration of biomolecules is below the certain predetermined concentration threshold;
storing a buffer in a scale buffer tank fluidly connected to the first column, and controlling with the monitoring and control unit the injection flow rate of the buffer from the scale buffer tank into the first column in order to maintain a certain filling level of the column according to the amount of reaction phase to be injected into the column, so that the lower the amount of reaction phase to be injected into the first column and the higher the amount of buffer to be injected into the first column;
passing, in several stages of an inline diafiltration system in fluid communication between a purification device and a lipid nanoparticles formulation system, the reaction phase with an exchange buffer, receiving in the monitoring and control unit data representative of a certain concentration of biomolecules to be used by the lipid nanoparticles formulation system to form the lipid nanoparticles, controlling with the monitoring and control unit the injection of the exchange buffer in the last stage in order to dilute the biomolecules at the certain concentration of biomolecules;
separating the manufactured biomolecules from the rest of the reaction phase with a purification system arranged at the outlet of the reaction chamber; and
measuring a certain concentration of each product in the rest of the reaction phase, and controlling with the monitoring and control unit the injection of the rest of the reaction phase in the mixing chamber.

### BRIEF DESCRIPTION OF THE DRAWINGS

The present disclosure will be better understood and other advantages will become apparent from the detailed description of the embodiment taken as a non-limiting example and illustrated by the attached drawings, in which:

FIG. 1 is a schematic representation of the system of the present disclosure;

FIG. 2 is a schematic representation of the first part of the system of the present disclosure used for the production of the biomolecules;

FIG. 3 is a schematic representation of the second part of the system of the present disclosure used for the purification of the molecules; and

FIG. 4 is a schematic representation of the third part of the system of the present disclosure used for the filtration of the biomolecules.

### DETAILED DESCRIPTION

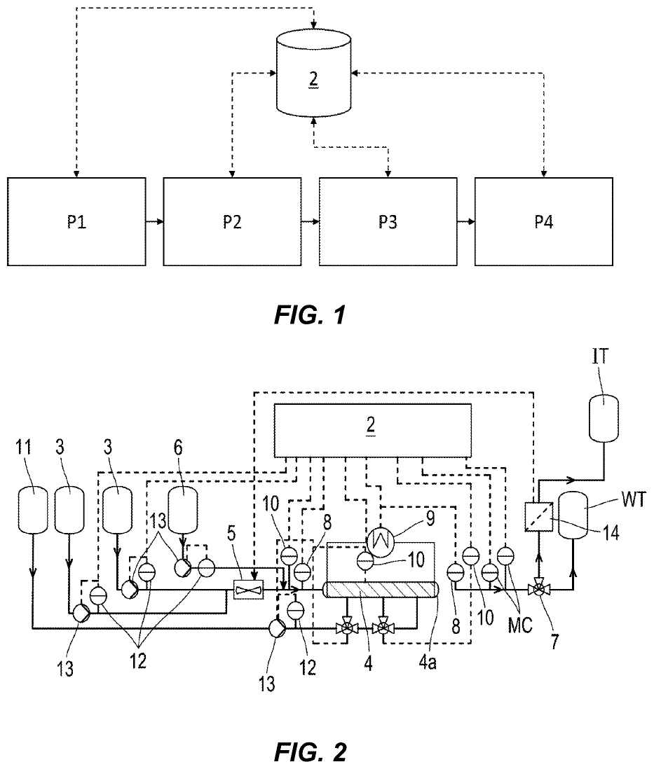

The system 1 for the continuous manufacture of biomolecules of the present disclosure, as shown in FIG. 1, is particularly adapted for producing different quantity and different type of biomolecules, such as RNA (Ribonucleic acid), DNA (Deoxyribonucleic acid) or proteins.

Those manufactured biomolecules could be used as a therapeutic agent, such as a vaccine.

The system 1 of the present disclosure works continuously notably thanks to a monitoring and control unit 2, which is set to enforce reaction conditions based on input parameterization and on a real time monitoring of the reaction conditions via a plurality of sensors distributed over the whole system for a feedback control of the reaction conditions according to the input parameterization.

In order to obtain the required quantity and quality of biomolecules, the reaction conditions that can be controlled in a non-exhaustive way are the temperature, the pH, the flow rate, the residence time, and the quantity of product to be injected for the reaction.

In the example as shown in FIG. 1, the system of the present disclosure could be divided in several parts each having a specific function in the manufacture of the biomolecules.

A first part P1 is dedicated to the production of the biomolecules.

The first part P1 comprises two feed tanks 3 suitable for storing products and a reaction chamber 4 designed to be fed with products from the feed tanks 3, which form in the reaction chamber 4 a reaction phase.

The reaction chamber 4, also called reactor or bioreactor, is here designed to produce the biomolecules from the reaction phase.

More particularly, the system 1 may comprise a mixing chamber 5 in fluid communication between the feed tanks 3 and the reaction chamber 4. In that case, the mixing chamber 5 is designed to be fed with products from the feed tanks 3 and to perform a mixing of the products to form the reaction phase and to feed in turn the reaction phase to the reaction chamber 4. The injection flow rate of the products from each feed tank is control in real time by a monitoring and control unit 2 set in order to maintain a constant ratio between the products in the mixing chamber 5. This allows to keep a constant reaction in spite of the change of the quantity of biomolecules to be manufactured. Indeed, the goal is to maintain a stable and reproducible reaction no matter the quantity of biomolecules to produce.

As shown in the FIG. 1, the system 1 comprises a scale tank 6 in fluid communication to the reaction chamber 4 and suitable for storing an immiscible phase, which is not miscible with the reaction phase.

The immiscible phase used in the system of the present disclosure can be liquid, such as an organic phase, solid or gaseous.

The monitoring and control unit 2 is thus set to control the injection flow rate of the immiscible phase into the reaction chamber 4 in order to keep constant a certain filling level of the reaction chamber 4 according to the amount of reaction phase to be injected into the reaction chamber 4 to manufacture a certain amount of biomolecules, so that the lower the amount of reaction phase to be injected into the reaction chamber 4 and the higher the amount of immiscible phase to be injected.

Moreover, in order to separate the immiscible phase from the reaction phase, the system could comprise a valve 7 at the outlet 4a of the reaction chamber 4. The monitoring and control unit 2 is thus set to receive a data representative of the presence of the reaction phase at the outlet 4a of the reaction chamber 4 and to control in turn the valve 7.

As, for example, the data representative of the presence of the reaction phase at the outlet 4a of the reaction chamber 4 is calculated based on a duration of a certain residence time of the reaction phase in the reaction chamber 4.

The reaction phase is thus injected into an intermediate tank IT and the immiscible phase is injected into a waste tank WT.

Without restricting the scope of the invention, the data representative of the presence of the reaction phase at the outlet 4a of the reaction chamber 4 can also be determined by UV sensors or RAMAN.

In order to maintain the reaction phase substantially at a predetermined temperature in the reaction chamber 4 according to the input parameterization, the system 1 may comprise, as shown in FIG. 2, a temperature sensor 8 designed to measure the temperature of the reaction phase, and a thermostat 9 arranged in contact with the reaction chamber 4 controlled by the monitoring and control unit 2 as a function of the measured temperature.

The system may also comprise a pH sensor 10 designed to measure the pH of the reaction phase. The system comprises, in that case, at least one buffer tank 11 fluidly connected to the reaction chamber 4 suitable to store a pH corrector product. The monitoring and control unit 2 is thus set to control the injection flow rate of the pH corrector product as a function of the measured pH level so as to maintain the reaction phase substantially at a predetermined pH level in the reaction chamber 4.

The system may also comprise mass flow meters 12 designed to measure the flow rate of each product to be injected in the reaction chamber 4. The system 1 comprises, in that case, a mass flow controller 13 designed to control the injection flow rate of these products in the reaction chamber 4. The monitoring and control unit 2 is thus set to control the mass flow controller 13 to control the injection flow rate of the reaction phase as a function of the measured flow rate so as to maintain the reaction phase at a certain flow rate in the reaction chamber 4.

The system 1 of the present disclosure may also comprise means MC for measuring the concentration of the biomolecules at the outlet 4a of the reaction chamber 4 obtained after a preset residence time in the input parameterization and in controlling in return the injection of the products into the reaction chamber 4 to increase the quantity of biomolecules produced to obtained the desired quantity of biomolecules.

The system 1 of the present disclosure may also comprise a purification system 14, here as example a filtration system, arranged at the outlet 4a of the reaction chamber 4 designed to separate the manufactured biomolecules from the rest of the reaction phase. In that case, the system may comprise means for measuring a certain concentration of each product in the rest of the reaction phase and the monitoring and control unit is thus set to control the injection of the rest of the reaction phase in the mixing chamber 5.

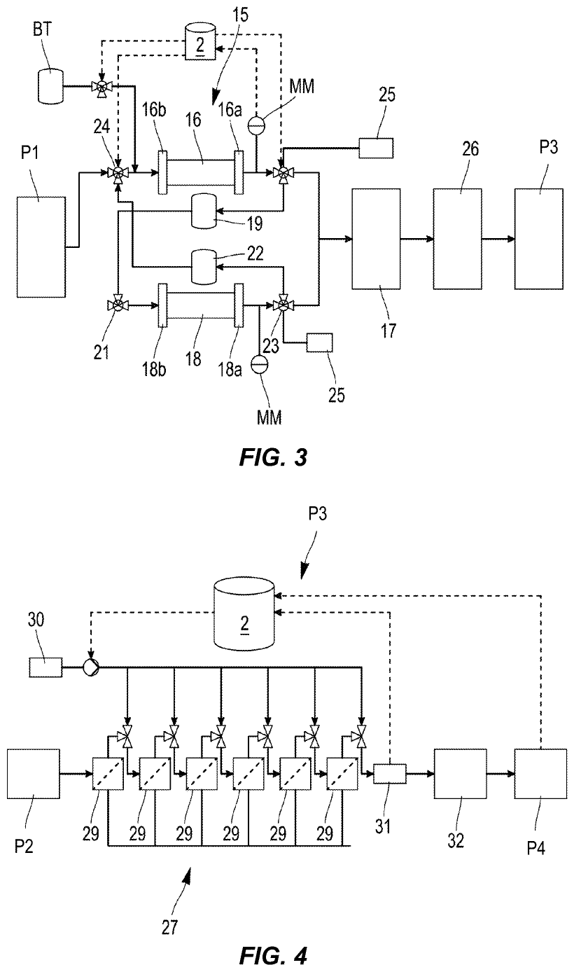

A second part P2 of the system 1 is dedicated to the purification of the biomolecules, as shown in FIG. 3.

In that purpose, the system of the present disclosure comprises a first chromatography device 15 comprising a first column 16 in fluid communication between the reaction chamber 4, and more particularly the intermediate tank IT, and a collection tank 17 and, a second column 18 in fluid communication between the first column 16 and the collection tank 17. The first chromatography device 15 further comprises a first temporary storage tank 19 in fluid communication between the first column 16 and the second column 18. The first chromatography device 15 further comprises a first outlet valve 20 at the outlet 16a of the first column 16 designed to switch the fluid communication from the first column 16 to the first temporary storage tank 19 or from the first column 16 to the collection tank 17, and a first inlet valve 21 at the inlet 18b of the second column 18 designed to open or close the fluid communication between the first temporary storage tank 19 and the second column 18.

More particularly, the first chromatography device 15 may comprise a second temporary storage tank 22 in fluid communication between the second column 18 and the first column 16. In that case, the first chromatography device 1 comprises a second outlet valve 23 at the outlet 18a of the second column 18 designed to switch the fluid communication from the second column 18 to the second temporary storage tank 22 or from the second column 18 to the collection tank 17, and a second inlet valve 24 at the inlet 16b of the first column 16 designed to open or close the fluid communication from the second temporary storage tank 22 to the first column 16.

The first chromatography device 1 may further comprise means for measuring the concentration of biomolecules at the outlet 16a, 18a of the first or second columns 16, 18 and the monitoring and control unit 2 is set to control the outlet valve 20, 23 at the outlet 16a, 18a of the first or second columns 16, 18 in order to feed, respectively, the first or second temporary storage tanks 19, 22 when the concentration of biomolecules in pre and post fractions of an eluted product fraction from the first or second column 16, 18 is above a certain predetermined concentration threshold or to feed a waste tank 25 when the concentration of biomolecules is below the certain predetermined concentration threshold.

A second chromatography device 26 can be used to improve the purification of the first chromatography device 1. As, for example, a reverse chromatography could be used.

A third part P3 is dedicated to the filtration of the purified biomolecules, as shown in FIG. 4, and a fourth part P4 is dedicated to the lipidic nanoparticles formation.

In this regard, the system 1 comprises an inline diafiltration system 27 in fluid communication between the first or second purification device 1, 26 and a lipid nanoparticles formulation system 28, the diafiltration system 27 comprising several stages 29 designed to pass the reaction phase with an exchange buffer stored in a buffer tank 30.

The monitoring and control unit 2 is thus set to receive data representative of a certain concentration of biomolecules to be used by the lipid nanoparticles formulation system 28 to form the lipid nanoparticles and a data of a real time concentration of the biomolecules measured by a concentration sensor 31 at the outlet of the last stage 29. The monitoring and control unit 2 is thus set to control the injection of the exchange buffer in the last stage 29 in order to dilute the biomolecules at the certain concentration of biomolecules and to inject the biomolecule at the certain concentration in an intermediate tank 32 before directing the biomolecules to the lipid nanoparticles formulation system 28.

The present disclosure further relates to a process for manufacturing biomolecules, here, for example, the manufacturing of mRNA (Messenger ribonucleic acid) for use as a vaccine.

A first step involves an in vitro transcription of mRNA. In this way, a first master mix comprising linearized specific DNA, coding for at least a part of one protein, is stored in the first feed tank 3 and the second master mix comprising RNA polymerase and nucleotides, is stored in the second feed tank 3. The first and second master mix are mixed in a chamber 5 at a specific ratio to form a reaction phase.

In order to maintain a constant ratio between both master mix in the mixing chamber 5, the injection flow rate of the products from each feed tank 3 is controlled in real time by the monitoring and control unit 2.

The reaction phase is then injected in the reaction chamber 4 to realize the in vitro transcription of the mRNA according to the reaction conditions imposed by an input parameterization of the monitoring and control unit 2.

This means that the reaction chamber 4 comprises all the necessary products and is able to provide all the necessary reaction conditions for the manufacture of mRNA.

The reaction chamber is also fed with an immiscible phase that is non miscible with the reaction phase, because of the reaction phase is an aqueous phase.

The monitoring and control unit 2 is thus set to control the injection flow rate of the immiscible phase into the reaction chamber in order to maintain a certain filling level of the reaction chamber 4 according to the amount of reaction phase to be injected into the reaction chamber 4 to manufacture a certain amount of biomolecules.

More particularly, the lower the amount of reaction phase to be injected into the reaction chamber 4 and the higher the amount of immiscible phase to be injected.

It follows that it is necessary to separate the immiscible phase from the reaction phase to enable quick and reliable purification of the mRNA produced.

To this end, the process of the present disclosure involves determining by way of the monitoring and control unit 2 and based on a certain duration of a certain residence time of the reaction phase in the reaction chamber 4, the presence or absence of the reaction phase at the outlet 4a of the reaction chamber 4, and controlling by way of the monitoring and control unit 2 the separation of the reaction phase from the immiscible phase.

There is also the need to reuse the products used in the reaction phase, such as enzymes and nucleotides, in order to limit production costs. For this purpose, the process involves separating the manufactured biomolecules from the rest of the reaction phase at the outlet 4a of the reaction chamber 4 by diafiltration.

An additional step involves in determining the concentration of each product of the rest of the reaction phase and of controlling in turn the injection of the rest of the reaction phase in the mixing chamber 5. The monitoring and control unit 2 is thus set to adjust the concentration of each master mix to be added from the feed tank 3 after the rest of the reaction phase to maintain a constant ratio.

The separated reaction phase is then purified in a chromatography step that includes separating a product fraction eluted from a first column 16 to a pre and post fractions of the product fraction eluted from the first column 16, directing the pre and post fractions from the first column 16 to a temporary storage tank 19, and directing the pre and post fraction from the temporary storage tank 19 to a second column 18 once both pre and post fractions are stored in the temporary storage tank 19.

The product fraction eluted from the second column 18 can thus be directed to a collection tank or purified again.

If the production fraction needed to be purified again, the process comprises an additional step that includes separating the product fraction eluted from the second column 18 to pre and post fractions of the product fraction eluted from the second column 18, directing the pre and post fractions from the second column 18 to a second temporary storage tank 22, and by directing the pre and post fraction from the second other temporary storage tank 22 to the first column 16 once both pre and post fractions are stored in the second temporary storage tank 22.

The idea is to elute a maximum of mRNA from the column by recovering the post and pre fraction without wasting time.

In that case, it is possible to measure the concentration of biomolecules at the outlet of the first or second column 16, 18 by way of measuring means MM, such as UV sensors, in order to direct the pre and post fraction from the first or the second columns, respectively, to the first or second temporary storage tank 19, 22 if the concentration is above a certain predetermined concentration threshold or to a waste tank 30 if the concentration is below the certain predetermined concentration threshold.

There is also the need to purify different amounts of biomolecules with the purification device 15 of the system 1 according to the present disclosure depending on the amount of biomolecules to be manufactured.

The idea of the present disclosure is thus to control the injection flow rate of a completing buffer in the first column 16 when the reaction phase passes in the first column 16 in order to maintain a certain constant level of filling in the first column 16.

In this situation, one single first column 16 can be used and dimensioned for the large quantity of biomolecules to be produced.

This allows continuous process by limiting the use of consumables and human intervention at the time of the change of scale of production.

By change of consumable, it should be understood the change of column according to the quantity of biomolecules to produce.

Thus, the monitoring and control unit 2 is set to control the injection flow rate of the completing buffer from a scale buffer tank BT of the chromatography device 15 into the first column 16 in order to maintain this certain filling level of the first column 16 according to the amount of reaction phase to be injected into the first column 16.

More specifically, this means that the lower the amount of reaction phase to be injected into the first column 16 and the higher the amount of buffer to be injected into the first column 16.

As for an example, the completing buffer used could be a neutral solution that does not modify the pH or the integrity and quality of the biomolecules produced.

It is to be understood, that the monitoring and control unit can be set to control the injection flow rate of the completing buffer from the scale buffer tank BT into the second column 18 to maintain a certain filling level.

A reverse chromatography could then be used in order to upgrade the quality of the purification.

A third step involves filtering the mRNA purified by the chromatography step.

This step includes filtering the mRNA of the reaction phase by passing the reaction phase with an exchange buffer in several stages 29 of a diafiltration system 27 and by injecting a certain amount of the exchange buffer in the last stage 29 in order to dilute the mRNA at a certain concentration ready to be used for the lipid nanoparticles formulation.

A fourth step involves the lipidic nanoparticles formation.

This step is known in its entirety, but the interest of the present disclosure is to be able to use a certain concentration of mRNA that is particularly well suited for the formation of nanoparticles. This concentration is determined by the input parameters and the dilution is performed during the diafiltration process.

1. A method of continuous manufacturing of biomolecules, comprising:
feeding a reaction chamber with products from feed tanks, the products forming a reaction phase in the reaction chamber;
storing an immiscible phase that is not miscible with the reaction phase in a scale tank in fluid communication with the reaction chamber;
controlling with a monitoring and control unit an injection flow rate of the immiscible phase into the reaction chamber to maintain a certain filling level of the reaction chamber according to an amount of reaction phase to be injected into the reaction chamber to manufacture a certain amount of biomolecules, so that the lower the amount of reaction phase to be injected into the reaction chamber and the higher the amount of immiscible phase to be injected; and
manufacturing the biomolecules from the reaction phase in the reaction chamber.

2. The method of claim 1, further comprising:
receiving, in the monitoring and control unit, data representative of a presence of the reaction phase at an outlet of the reaction chamber; and
controlling in turn, with the monitoring and control unit, a valve at the outlet of the reaction chamber for separating the immiscible phase from the reaction phase.

3. The method of claim 2, wherein the data representative of the presence of the reaction phase at the outlet of the reaction chamber is calculated based on a duration of a residence time of the reaction phase in the reaction chamber.

4. The method of claim 3, further comprising:
feeding a mixing chamber in fluid communication between the feed tanks and the reaction chamber with products from the feed tanks;
performing a mixing of the products to form the reaction phase and feeding in turn the reaction phase to the reaction chamber; and
controlling, with the monitoring and control unit, the injection flow rate of the products from each feed tank in real time to maintain a constant ratio between the products in the mixing chamber.

5. The method of claim 1, further comprising performing chromatography including:
separating a product fraction eluted from a first column to a pre and post fractions of the product fraction eluted from the first column;
directing the pre and post fractions from the first column to a first temporary storage tank; and
directing the pre and post fraction from the first temporary storage tank to a second column once both pre and post fractions are stored in the temporary storage tank.

6. The method of claim 5, further comprising:
separating a product fraction eluted from the second column to a pre and post fractions of the product fraction eluted from the second column;
directing the pre and post fractions from the second column to a second temporary storage tank; and
directing the pre and post fraction from the second temporary storage tank to the first column once both pre and post fractions are stored in the second temporary storage tank.

7. The method of claim 6, further comprising:
measuring a concentration of biomolecules at the outlet of the first column or the second column; and
controlling, with the monitoring and control unit, the outlet valve at the outlet of the first column or the second column to feed, respectively, the first temporary storage tank or the second temporary storage tank when the concentration of biomolecules in pre and post fractions of an eluted product fraction from the first column or the second column is above a certain predetermined concentration threshold or to feed a waste tank when the concentration of biomolecules is below the certain predetermined concentration threshold.

8. The method of claim 7, further comprising:
storing a buffer in a scale buffer tank fluidly connected to the first column; and
controlling, with the monitoring and control unit, the injection flow rate of the buffer from the scale buffer tank into the first column to maintain a certain filling level of the column according to the amount of reaction phase to be injected into the column, so that the lower the amount of reaction phase to be injected into the first column and the higher the amount of buffer to be injected into the first column.

9. The method of claim 1, further comprising:
passing, in several stages of an inline diafiltration system in fluid communication between a purification device and a lipid nanoparticles formulation system, the reaction phase with an exchange buffer;
receiving, in the monitoring and control unit, data representative of a certain concentration of biomolecules to be used by the lipid nanoparticles formulation system to form the lipid nanoparticles; and
controlling, with the monitoring and control unit, the injection of the exchange buffer in the last stage to dilute the biomolecules at the certain concentration of biomolecules.

10. The method of claim 1, further comprising separating the manufactured biomolecules from the rest of the reaction phase with a purification system arranged at the outlet of the reaction chamber.

11. The method of claim 10, further comprising:
feeding a mixing chamber in fluid communication between the feed tanks and the reaction chamber with products from the feed tanks;
performing a mixing of the products to form the reaction phase and feeding in turn the reaction phase to the reaction chamber;
controlling, with the monitoring and control unit, the injection flow rate of the products from each feed tank in real time to maintain a constant ratio between the products in the mixing chamber;
measuring a certain concentration of each product in the rest of the reaction phase; and
controlling, with the monitoring and control unit, the injection of the rest of the reaction phase in the mixing chamber.

12. The method of claim 1, wherein the manufactured biomolecules are RNA.

13. The method of claim 12, wherein the RNA manufactured is mRNA.

14. The method of claim 1, wherein the manufactured biomolecules are proteins.

15. The method of claim 1, wherein the manufactured biomolecules are DNA.

16. The method of claim 1, wherein the manufactured biomolecules are a therapeutic agent.

17. The method of claim 16, wherein the therapeutic agent is a vaccine.

18. The method of claim 1, further comprising:
feeding a mixing chamber in fluid communication between the feed tanks and the reaction chamber with products from the feed tanks;
performing a mixing of the products to form the reaction phase and feeding in turn the reaction phase to the reaction chamber; and
controlling, with the monitoring and control unit, the injection flow rate of the products from each feed tank in real time to maintain a constant ratio between the products in the mixing chamber.

19. The method of claim 5, further comprising:
storing a buffer in a scale buffer tank fluidly connected to the first column; and
controlling, with the monitoring and control unit, the injection flow rate of the buffer from the scale buffer tank into the first column to maintain a certain filling level of the column according to the amount of reaction phase to be injected into the column, so that the lower the amount of reaction phase to be injected into the first column and the higher the amount of buffer to be injected into the first column.
